(19) United States
(12) Patent Application Publication
(10) Pub. No.: US 2020/0268611 A1
(43) Pub. Date: Aug. 27, 2020

(54) CONTAINER CAP WITH MODULAR ELECTRONIC SYSTEM AND METHOD

(71) Applicant: KING ABDULLAH UNIVERSITY OF SCIENCE AND TECHNOLOGY, Thuwal (SA)

(72) Inventors: Muhammad Mustafa HUSSAIN, Austin, TX (US); Sherjeel KHAN, Thuwal (SA)

(21) Appl. No.: 16/634,784

(22) PCT Filed: Aug. 20, 2018
(86) PCT No.: PCT/IB2018/056294

§ 371 Date: Jan. 28, 2020

Related U.S. Application Data
(60) Provisional application No. 62/572,915, filed on Oct. 16, 2017.
(60) Provisional application No. 62/700,664, filed on Jul. 19, 2018.

(51) Int. Cl. A61J 7/00; A61J 7/02; B65D 83/04; B65D 51/24; G16H 40/67

(52) U.S. Cl. CPC A61J 7/0076; A61J 7/02; B65D 83/0409; G16H 20/13; G16H 40/67; A61J 2200/72; A61J 2200/70; B65D 51/24

(57) ABSTRACT

A control system for monitoring drug dispensation from a container. The system includes a base cap configured to be attached to the container; an electronic interface attached to the base cap; a microprocessor attached to the electronic interface; a pill counter mechanism attached to the base cap and configured to count pills that are passing the base cap; and a top lid that attaches to the base cap and fully encloses the electronic interface, the microprocessor and the pill counter mechanism.

### CROSS REFERENCE TO RELATED APPLICATIONS

This application claims priority and benefit from U.S. Provisional Patent Application No. 62/572,915, filed on Oct. 16, 2017, for “Modular Electronic System for Safe-Administering of Prescription Drugs,” and U.S. Provisional Patent Application No. 62/700,664, filed on Jul. 19, 2018, for “Container Cap with Modular Electronic System and Method,” the contents of which are incorporated in their entirety herein by reference.

### BACKGROUND

### Technical Field

Embodiments of the subject matter disclosed herein generally relate to electronic systems for controlling the dispensation of a material from a container, design of the electronic cap to house the pill counter and more specifically, to methods and flexible electronic systems housed inside a cap of a container for monitoring drug traffic through the cap.

### Discussion of the Background

Over the past three decades, a global epidemic of prescription drug abuse has been on the rise. Although there are many attempts to solve this matter, there is no known tangible solution to this epidemic. The global estimate for drug-related deaths in 2014 was over 200,000 despite the fact that drug addiction usage has been stagnant for 10 years. This points to a more serious problem: that these deaths are correlated to a growing rise in prescription drug abuse, not drug addiction. U.S.A. was hit the hardest with a drug overdose death toll rising from 6,100 in 1980 to 47,055 in 2014, which is nearly 125 people dying every day. Half of these deaths have directly been attributed to prescription drug overdoses. Along with the increased use of opioids for medicinal purposes, their use for non-medicinal purposes has been on the rise as well. A study conducted by National Survey on Drug Use and Health found that, only in the U.S.A. alone, about 4.8% individuals consumed analgesics between the years 2002 to 2005 for nonmedical purposes. Even at times when statistics showed a decrease in deaths caused by drug overdose, the numbers for prescription opioid use disorders have been ever increasing. Additionally, U.K. saw an increase by 4.8% in the decade since 2004 while Canada suffered from an 80% annual increase of drug-related deaths since 2010. Overdose drug-related deaths have been spreading across Europe where a 6% increase was observed in 2016 compared to 2015.

Experts predict that soon, the annual number of deaths in the U.S.A. due to drug overdose would exceed breast cancer deaths, which clearly indicates that this issue needs to be given as much attention as cancer. Stringent state laws to control this problem seems like a probable solution. However, limiting access of opioid pain relievers to people may result in an increase in mortality rates because people start looking for more dangerous alternative drugs to treat their pain.

Taking drugs without doctor supervision presents even greater chances of a possibly fatal drug overdose. When rejected, by a doctor and/or pharmacy, the patients hop from doctor to doctor and pharmacy to pharmacy to have their prescription containers filled, and a study found that these shoppers are more prone to end up with a drug overdose-related death. The U.S. government has implemented a Prescription Drug Monitoring Program (PDMP) in more than 40 states to monitor prescription drug use for isolating suspicious activities. Studies conclude that PDMPs did not cause any reduction in drug overdose, but may in fact have sparked an increased usage of illegal drugs as both PDMP and non-PDMP states showed the same kind of rise in drug overdose-related deaths. Another study indicates that several existing methods of drug overdose adherence monitoring/prevention alone, have all proved to be ineffective including: prescription monitoring programs, screening tools to monitor opioid adherence or urine drug testing.

The existing methods for screening potential drug abusers seem ineffective. Relying solely on methods like prescription drug monitoring program, controlled substance laws, shutting down pharmacies, patient and physician educational initiatives, law enforcement initiatives on patients and clinics, promoting compassionate practices among physicians, and lowering the recommended drug threshold, has not been able to resolve the overdose issue.

Thus, another approach is the use of electronics for monitoring the drug consumption. However, traditional Printed Circuit Board (PCB) based integration strategies offer little to no customizability or modularity due to high costs associated with making a diverse range of products. There are several pill dispensers available in the market, like Philips Lifeline®, Medminder™, Hero® and LiveFine™, but their persistent challenge is either a bulky form factor, an expensive price tag, or lack of any smart features. Further, these products require patients to carry an extra item with them at all times. Humans tend to prefer convenience over utility, and thus the proliferation of such pill dispensers is believed to be limited in future.

Looking at the current circumstances and failure of the existing systems to counteract the overdose problem, a smarter way needs to be developed to control and/or monitor the drug consumption for medically prescribed drugs.

### SUMMARY

According to an embodiment, there is a control system for monitoring drug dispensation from a container. The system includes a base cap configured to be attached to the container, an electronic interface attached to the base cap, a microprocessor attached to the electronic interface, a pill counter mechanism attached to the base cap and configured to count pills that are passing the base cap, and a top lid that attaches to the base cap and fully encloses the electronic interface, the microprocessor and the pill counter mechanism.

According to another embodiment, there is a drug dispensing system that includes a container having an open end; a base cap configured to be attached to the container and close the open end; a control system located on the base cap; and a top lid attached to the base cap and covering the control system. The control system is configured to monitor a pill entering or leaving the container.

According to still another embodiment, there is a method for dispensing a drug, the method including providing a container having an open end; attaching a base cap to the container to close the open end; locating a control system on the base cap; attaching a top lid to the base cap to cover the control system; and monitoring a pill entering or leaving the base cap with the control system.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 illustrates a pill dispensing system;

FIGS. 2A to 2D show a base cap and a top lid of the pill dispensing system;

FIGS. 3A to 3C illustrate the base cap, a pill counter mechanism and an alerting device;

FIGS. 4A to 4D illustrate how the pill counter mechanism counts the pills moving in and out of a container;

FIGS. 5A to 5B illustrate how a strain sensor is attached to the container;

FIGS. 6A to 6B illustrate how the control system is placed on the base cap and under the top lid;

FIGS. 7A to 7E illustrate how the various components of the control system are added to an electronic interface and then to the base cap;

FIGS. 8A to 8F illustrate how the paper based temperature and humidity sensors are converted to modular form and attached to a flexible substrate;

FIG. 9 schematically illustrates a block diagram of the internal features of the microprocessor that manages all the sensors added to the control system; and

FIG. 10 is a flowchart of a method for assembling a pill dispensing system.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a prescription drug dispensed from a plastic bottle and having a cap. Those skilled in the art would understand that the teachings of the following embodiments may be applied to other type of containers and for other substances.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, it is possible to use low-cost paper and stretchable sensors together with a central controller to implement an affordable self-contained pill monitoring device that counts pill usage and reports, for example, using a cell phone, if overuse or tampering of the container is taking place. The problem with the existing pill containers is that once the pharmacy sells the drug to the patient, the use of the drug is no longer under anyone's control, except for the patient's. Patients may forget to take the pill, or may suffer from intentional or non-intentional drug overdose. To overcome this issue, the following embodiments propose a way to incorporate an add-on system to the standard prescription container. This system may count and record pill usage, and then syncs the data through the patient's cell phone with a remote server (e.g., the pharmacy's server), allowing the patients to properly follow the recommended dosage.

For patients suffering from a drug overdose disorder, the device can generate an alert in an event that more than the recommended pills for a certain time period are dispensed from the container. According to such a system, the pharmacies may have a log of all these activities so that when the patient returns for a refill, they can have a substantial and reliable screening method to decide whom to give the pills for. Because the functionality of this new system is kept modular, paper-based temperature and humidity sensors (or any other sensor) can be attached as needed to monitor the ambient environment inside the prescription container. Additionally, with the addition of some smart modules, the container will have the ability to provide approximate location of the patient and inform authorities or caretakers via SMS/Call whenever the patient is tampering with the container or abusing the recommended dosage. This feature allows for an immediate on-demand response that can potentially save the patient from imminent death.

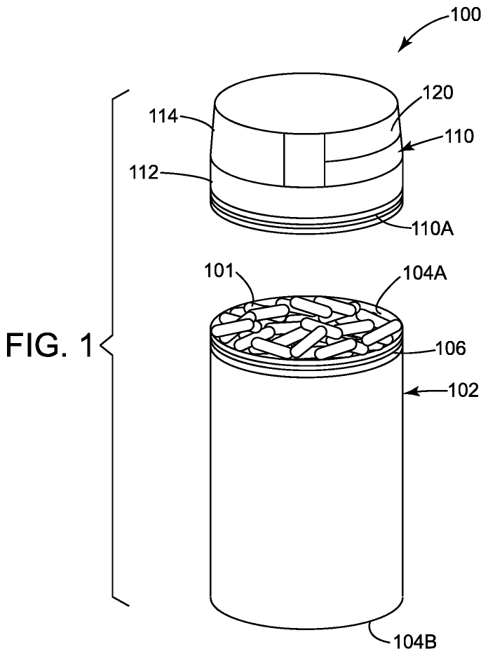

According to an embodiment, FIG. 1 illustrates a system 100 for monitoring and/or controlling the flow of pills 101 in and out of a container 102. The container may be any container. For example, container 102 may be a typical pill container used by pharmacies in U.S.A. Such a container may be cylindrical and may have an open end 104A and a closed end 104B. An exterior part of the container 102, adjacent to the open end 104A, may include threads 106 that mate with corresponding threads 110A of a cap 110. Cap 110 may be made of two parts, a base cap 112 that directly attaches to the container 102 and closes the open end 104A and a top lid 114 that attaches to the base cap 112. The top lid 114 may be glued to the base cap 112 or attached with other means (e.g., threads). A control system 120 may be located between the base cap 112 and the top lid 114, as now discussed.

The base cap 112 and top lid 114 may be made of plastic. In one application, these caps may be made with a 3D printer. The design of the base cap still maintains the child-proof capabilities of original caps, including the twist and lock feature. The control system 120 uses flexible sensors and simplified minuscule electronic interface so that the whole system can fit on the cap without adding excessive weight, complexity, and cost. As discussed later, the control system has a communication device that can communicate, for example, via a mobile phone of the user of the container, with the current pharmaceutical chain. By having a low-cost and readily replaceable cap that can be easily attached to the existing prescription containers, it is believed that such a system has the potential for widespread adaptation, which will help save thousands of lives in the future.

Customization can be effective to reduce design complexity and power consumption while delivering features as per the necessity of different situations. Therefore, one advantage of using modular decal (i.e., paper thin) sensors is that the shape and functionality of the entire system can be molded as per the application requirement. When security is desired, safety sensors can be attached to the control system 120. When monitoring pill integrity is important, paper-based sensors can be attached to the control system 120. The assembling and fabrication cost of such sensors is so low that the sensors can be used as disposable sensors. The combined cost of paper sensors, pill counter, and security sensors for such a system are anticipated to be less than $2.

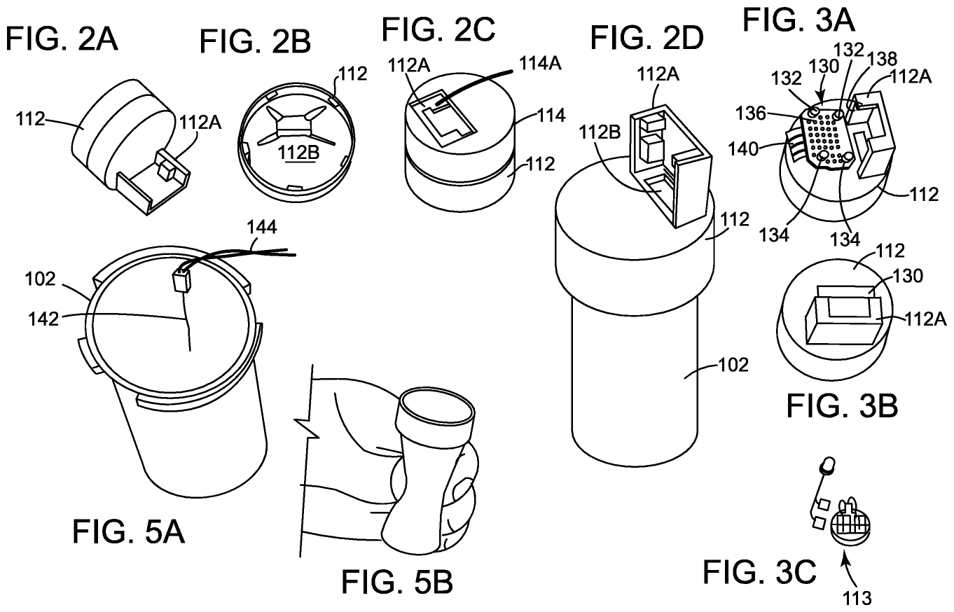

The base cap 112 shown in FIG. 1 is similar to a conventional prescription container, but, as shown in FIG. 2A, has been customized to carry the control system 120 and various sensors (to be discussed) without hindering the usage of the container 102. For this embodiment, the base cap 112 was printed using a 3D Printer (Leapfrog™ XEED 3D Printer), maintaining the twist and lock feature (child-lock) of normal prescription containers. The side and bottom views of the base cap 112 are shown in FIGS. 2A and 2B, respectively. Note that the base cap 112 has a slide 112A through which the pills are allowed to move. The base cap also has a slit-like opening 112B, which communicates with the slide 112A to allow the pills to come out of the container. The slide 112A houses the pill counter mechanism 130 (which is discussed later) shown in FIGS. 3A to 3C.

The top lid 114, shown in FIG. 2C, is attached to the base cap 112 so that a slit 114A in the top lid 114 comes directly over the slide 112A entrance of the cap 112. The base cap 112, without lid 114, is shown in FIG. 2D being attached to the container 112. In one application, the cap 112 together with the lid 114 adds only 2.2 cm of height compared to a conventional cap, thus not affecting the accustomed usage of the original prescription containers. The prescription container with this customized cap can still easily fit in a pocket or a purse.

One component of the control system 120 is a pill counter mechanism 130. One function of the control system 120 is to reliably count the number of pills coming out and going into the container. The base cap 112 was designed in such a way that pills can be accessed by tilting the bottle and shaking so the pills come out of the slider 112A one at a time. As the control system 120 is intended to keep a log of all the pills coming out of the container and detect any anomaly, the pill counter mechanism 130 may include, in one embodiment, two sets of (i) an Infrared Light Emitting Diode (IR LED) and (ii) a photodiode (each set including one IR LED and one photodiode) which fit snugly inside the grooves 138 on the slider 112A of the base cap 112, as shown in FIGS. 3A and 3B. One skilled in the art would understand that other types of pill counter mechanisms may be used. The one discussed herein has been selected for being one of the simplest and cheapest to make.

The two IR LEDs 132 and the two photodiodes 134 are placed in front of each other on a board (e.g., Veroboard) 136, as illustrated in FIG. 3A. They are spaced apart on the board 136 according to the width of the slider 112A so that the LEDs and photodiodes fit into the 4 grooves 138 on the sides of the slider 112A, as shown in FIG. 3A. An additional LED 113, see FIG. 3C, may be added to the cap for providing an alert to the user of the container. This feature will be discussed later in more detail.

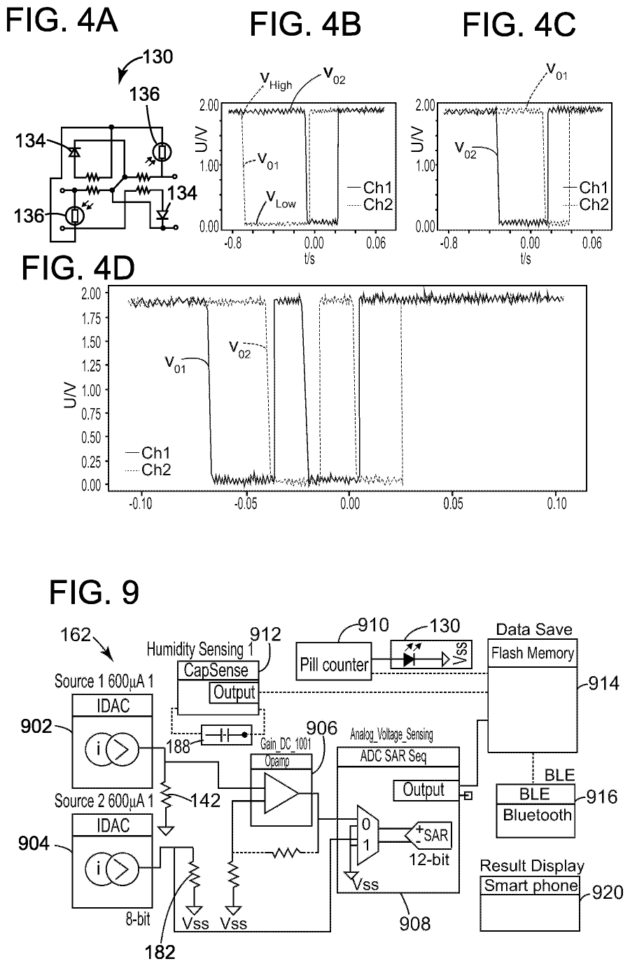

A schematic of the pill counter mechanism 130 is shown in FIG. 4A. This schematic shows four outputs coming out of the pill counter mechanism 130, Power Vcc, Ground GND, Vo1 (Output Voltage 1), and Vo2 (Output Voltage 2). The two output voltage nodes are used to monitor if a pill passes between the IR LED and a photodiode. When there is no obstruction between a pair of an IR LED and a photodiode, the output of the pill counter mechanism 130 is a high voltage Vhigh (see FIG. 4B) because the IR LED keeps the photodiode on, and a large current passes through the resistor R in series with the photodiode. As soon as something comes in between the IR LED and photodiode, the voltage output drops down (see Vlow in FIG. 4B) as the current passing through the photodiode is reduced due to IR rays not reaching the photodiode. Due to the lower current passing through the series resistor R, the voltage level drops.

FIG. 4B shows high and low values for two voltages, Vo1 and Vo2. The reason for using two sets of IR LEDs and photodiodes is to recognize the direction of the pill coming out or going into the bottle. For example, FIG. 4B illustrates the voltages for Channel 1 and Channel 2, wherein Channel 1 corresponds to the IR LED and the photodiode on the right in FIG. 4A and Channel 2 corresponds to the one on the left. If a pill is going into the slider, Channel 2 (Vo1) will go low first and then Channel 1 (Vo2) will go low as shown in FIG. 4B. If a pill is coming out of the slider, Channel 1 (Vo2) will go low first and then Channel 2 (Vo1) will go low as seen in FIG. 4C.

There may be a situation when two pills are coming out side by side from the slider. For this situation, it is desired that the device counts them properly. This demands a fast response from the optocouplers in the pill counter mechanism. Pills always have shapes with different dimensions at the center and at the end. Thus, this feature enables the pill counter mechanism of this embodiment to detect the end of one pill before the start of the second pill, even when two pills are placed side by side in the slider.

For example, FIG. 4D shows the output of the pill counter mechanism when two pills are coming out of the bottle right next to each other. It can be seen in FIG. 4D that there is a 10 ms of high output voltage for both channels, between the two low voltage depression of each channel (low voltage indicates pill presence, while high voltage indicates pill absence when passing between the IR LED and photodetector of the pill counter mechanism). The electronic interface (discussed later) was designed in such a way that it reads the pill counter mechanism output every 1 millisecond.

Thus, in this embodiment, the pill counter mechanism is able to differentiate between two pills coming out even when placed side by side. This is important for the case in which the patient takes out 2 pills at a time by mistake. The software then allows the patient to put back into the bottle the additional pill before an alert is generated. The software will also log the number of pills being added into the bottle at the pharmacy, so that the system can keep a count of the number of pills in the bottle at all times.

Returning to FIG. 3A, there are four interconnections 140 attached to the board 136, to maintain the modularity of the control mechanism 120 and to attach the control system 120 to the electronic interface. These four interconnects may be made on a Polyimide sheet, as discussed later. Copper tape interconnects may be soldered to each of the four outputs from the pill counter mechanism. More or less interconnections may be used.

As the control system 120 is keeping track of the number of pills coming out and going into the container through the cap, it is necessary to make sure that there are no other means available to remove the pills from the container. As discussed above, the pill counter mechanism controls the ingress and egress through the cap slider.

There are two other ways a patient can remove a pill from the container without engaging the pill counter mechanism: (1) remove the cap from the container and/or (2) break the container. To detect an attempt of breaking the container, it is possible to use a strain sensor (e.g., a conductive rubber cord stretch sensor) 142, as illustrated in FIG. 5A. The resistance of the strain sensor 142 changes when the sensor is stretched or bent. This change in resistance can be detected by the electronic interface and used to generate an alert.

The stretchable sensor may be housed inside a layer of Polydimethylsiloxane (PDMS) and attached to the inner walls of the container 102, as illustrated in FIG. 5A. To make contacts with the strain sensor, stainless steel conductive 144 fibers are attached at both ends with a knot. This arrangement provides good contact to the strain sensor, so it can be attached to the electronic interface. PDMS helps the sensor to be attached inside the container while allowing it to stretch.

When a patient tries to break the container 102 as illustrated in FIG. 5B, the strain sensor 142 bends and the electronic interface generates an alert based on the change in the strain sensor's resistance. Similarly, if a patient removes the cap, the strain sensor is stretched laterally as the contacts 144 from the strain sensor 142 are going to the electronic interface, which is situated on the cap. The stretching action produces a change in resistance, which can again be identified by the electronic interface to generate an alert. In case that the contact or the strain stretch sensor breaks, the connection will in turn be broken, which is also sensed by the electronic interface to generate an alert.

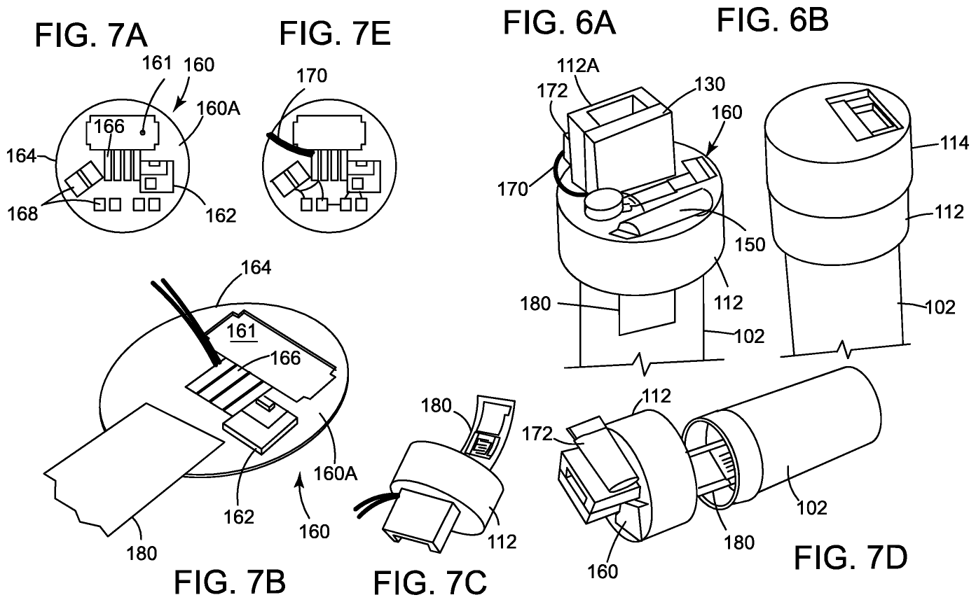

The control system 120 includes the electronic interface, which connects to all the previous elements (e.g., pill counter mechanism, strain sensor) and acts as the brain for recording all the activities and sending one or more alarms as the case is. If someone tampers with the electronic interface, the purpose and functionality of the entire control system 120 is compromised. To prevent mishandling of the electronic interface, in one embodiment, a tamper sensor 150, as shown in FIG. 6A may be placed over the electronic interface 160. The tamper sensor 150 may be made as a paper-based sensor. The tamper sensor is a proximity based pressure sensor to not only detect if someone touches the control system 120, but also can identify when a hand comes in close vicinity of the electronic interface. The tamper sensor 150 may be developed using the techniques discussed in Nassar et al. work on paper-based sensors by making a parallel-plate capacitive sensor using flexible aluminum foil for the metal plates, and a microfiber wipe placed in-between to serve as the pressure sensitive dielectric.

When pressure is applied to the tamper sensor 150, the microfiber wipe compresses, changing the spacing between the two plates, which translates into a change in capacitance, which may be detected by a microcontroller (to be discussed later). This sensor may also be converted into a modular decal sensor which can then be integrated with the central electronic interface 160. The tamper sensor sticker 150 is attached directly above the electronic interface, as shown in FIG. 6A, to successfully log any attempt of approach or mishandling. While FIG. 6A shows the control system 120 formed on the base cap 112, FIG. 6B shows the top lid 114 placed over the control system 120 and shielded from the ambient.

To generate an alert, one or more components may be used depending upon the application. If the microcontroller possesses Bluetooth functionality, an electronic alert can be generated on the patient's mobile phone at any times. Instead or in addition, a buzzer or LED 113 (see FIG. 3C) may be attached to the cap in a modular fashion. For example, to indicate the correct number of pills coming out, the LED 113 can be used. The electronic interface may be programmed to turn the LED on when the time to take the pill has arrived. Once the right number of pills have been taken out of the container, the LED will be turned off, and if extra pills are removed from the container, the LED starts blinking. Similarly, the frequency and intensity of the buzzer can be used to indicate the aforementioned events.

The central electronic interface 160 can be made to be very thin, similar to the sensors discussed above, i.e., the decal sensors. In this regard, an integration approach (see U.S. Pat. Nos. 9,209,083 and 9,520,293) may be used to make any desired decal system for a specific application in modular and customizable fashion. To make a fully conformal and flexible electronic interface for applications that demand complete flexibility, it is possible to use flexible paper-based sensors. Other than using the flexible paper-based sensors demonstrated by Nassar et al., Rojas et al. and Torres Sevilla et al. have successfully demonstrated how bulk monocrystalline Silicon (100) based high-performance advanced Complementary Metal Oxide Semiconductor (CMOS) devices and circuitry can be flexed down to 0.5 mm bending radius.

Because for this application it is desired to embed the control system inside the prescription container, a fully flexible decal electronic interface is desired. As illustrated in FIG. 7A, a flexible microprocessor 162 was attached to a substrate 164 (of the electronic interface 160) made of, for example, polyimide sheet. Electrical connections 166 were made to the copper interconnects on the back side through-polymer-vias. Other electrical connections 168 are shown and they are used for connecting the pill counter mechanism and any other sensor that may be used. An environment sensor (paper temperature and humidity sensors) 180, to be discussed later, is attached to the flexible electronic interface 160 as shown in FIGS. 7B and 7C. The whole environment sensor 180 was then lined inside the prescription container 102 such that it clasps the inner walls of the container, as shown in FIG. 7D. Thus, this arrangement can monitor the temperature and humidity inside the container. Because of the compact, lightweight and flexible property of the control system, day to day usage of the prescription container is rendered easy to carry, while providing the patient with such an advantageous utility.

In one application, to control system 120 has the capability of wireless data logging and transmission. This may be achieved, for example, with a Programmable System on Chip (PSoC) system. Such a PSoC system may be a Cypress® BLE PSoC, which is used as the brains of the electronic interface sticker. This PSoC possesses wireless functionality in the form of Bluetooth Low Energy (BLE) network so it can communicate with a mobile phone to generate alerts. The PSoC system may also contain, in addition to a processor, a 256 kB flash memory, to log pill intake data even at times when the control system is not connected to a smartphone.

The electronic interface 160 shown in FIG. 7A was made as now discussed. A Polyimide sheet (substrate 164) was cut in such a way that it fits on the top of the base cap 112, as shown in FIG. 6A. An opening 161 has been cut into the substrate 164 to accommodate the slide 112A of the base cap 112. The footprint of the electronic interface 160 was created using conductive copper tapes in such a way that it replicated the shape of interconnects on the environment sensor 180, pill counter decal mechanism 130, and the buzzer 113. On the top side 160A of the electronic interface 160, the environment sensor 180 was attached to its respective site, as shown, for example, in FIG. 7B. The copper tape making the contacts 166 went all the way to the back of the substrate 164, through the slit 161 and connections were made between PSoC 162 and copper tape interconnects by soldering wires between them on the back side. A JST 2-plug wire 170 was soldered on the PSoC power terminals so that the whole system could be easily powered using a 3.7V rechargeable Li-ion battery as shown in FIG. 7E.

A Z-axis conductive tape (3M Z-Axis Conductive Tape 9703) was placed on the whole decal area where the copper tape interconnects 168 are present. The transparent Z-axis conductive tape has small gold granules, which allow for anisotropic conduction of electric current. The environment sensor 180 is then attached to its respective site on the electronic interface decal 160 by simply aligning the interconnects on both decals, and pressing the paper sensor module 160 for a few seconds, as illustrated in FIG. 7B. The stickers are then electrically connected to each other through the Z-axis conductive tape. This process connected the environment sensor 180 to the electronic interface 160 with a small alignment and finger pressure in a very simplistic way. The contact resistance is negligible (˜1Ω). Similarly, the pill counter mechanism 130 and the buzzer 113 were attached to their respective sites as shown in FIGS. 7D and 6A.

The control system 120 was then placed on top of the base cap 112, as shown in FIG. 6A, such that the pill counter mechanism 130 resides in slide 112A, and the environment sensor 180 was passed through the cap through a slit such that it is housed inside the container 102. FIG. 6A also shows the tamper sensor 150, and battery 172 connected to wires 170. Owing to the customizability and modularity of the process discussed above, and the use of flexible sensors and substrate, the whole control system 120 seamlessly merges together with a smaller footprint such that the lid 114 can easily be placed on top of the base cap 112 to conceal the whole system, as shown in FIG. 6B. The battery 172 can be attached to the back side of the slider 112A and connected to the JST-2 pin power socket 170 to power up the whole system, as shown in FIG. 7D.

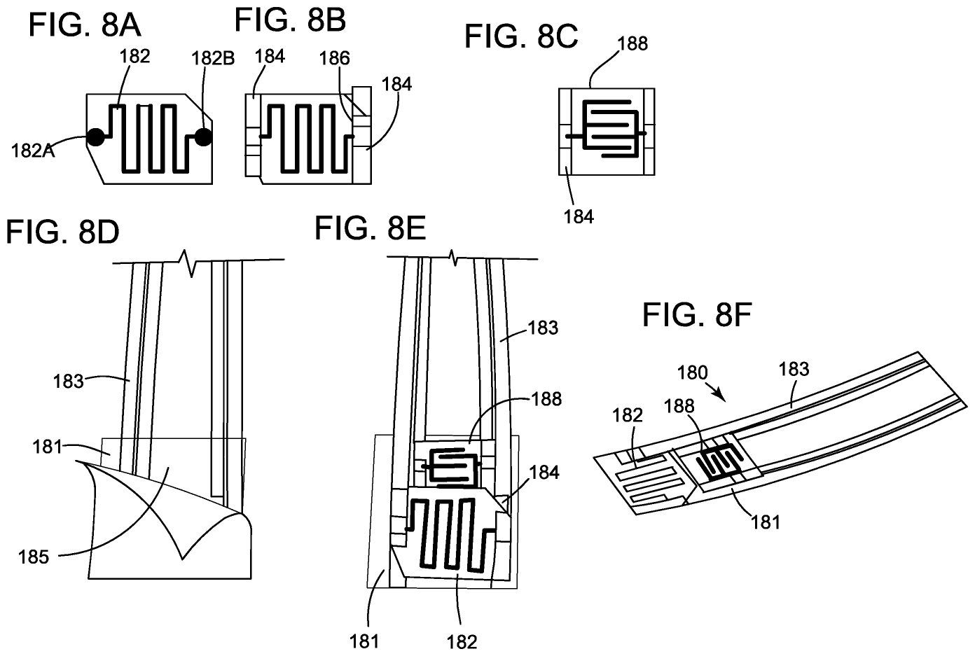

The environment sensor 180 is now discussed with regard to FIGS. 8A-8F. Storage conditions are vital for the integrity and effectiveness of most medicines. Temperature and humidity are known to influence the integrity and potency of medicinal pills. Elevated temperatures and humidity increase the degradation time of tablets. Doctors recommend storage of most medicines at room temperature. For example, any tablet containing chitosan has to be stored under 25° C. and 60% RH (Relative Humidity) to avoid worsening of its physical properties. Similarly, for Ascorbic Acid based tablets, which are the most widely used for counteracting Vitamin C deficiency, studies show that increased temperature and humidity severely affect their stability. Several studies have shown a severe decrease in dissolution time of tablets under exacerbated humid conditions, where moisture absorption can also change the appearance, hardness, mass and crystallinity of the medicine. This makes it desirable to monitor the storage conditions inside the prescription container as they are more prone to deterioration once removed from foil.

Nassar et al. have previously demonstrated the performance characteristics of paper-based humidity and temperature sensors for wearable applications. Those sensors showed consistent performance under various bending conditions, which is a suitable attribute for this application. Thus, in one embodiment, it is possible to use paper-based sensors with the purpose of monitoring the ambient environment inside the container. The paper-based temperature sensors are shown to deliver linear results from 20-100° C. Because a goal of using these sensors is to monitor the ambient temperature inside the container, such a temperature range is suitable for this application.

To integrate paper-based sensors on a flexible platform as the substrate 164 in FIG. 7A, gold or copper interconnect patterns are deposited on the flexible polyimide substrate. The environment sensor is connected to the interconnects using epoxy or other conductive adhesives. This increases the cost and complexity of the procedure due to the use of sophisticated equipment in cleanroom facilities.

Thus, another approach was to convert paper sensors into modular decal sensors that can be attached to the electronic interface 160 in any customized fashion. A method for converting paper sensors into modular form is now discussed with regard to FIGS. 8A to 8F. A paper temperature sensor 182 was made on a paper substrate as shown in FIG. 8A. The temperature sensor 182 includes a strip of copper. Separately, conductive copper tape was cut into thin long strips 184 and then connected to electrodes 182A and 182B of the paper temperature sensor 182, as shown in FIG. 8B. The strips 184 are shown in FIG. 8B being attached to one end of the electrodes, and then folded towards the backside of the sensor, enabling later the modular attachment of the sensor to a base sticker 164 (in FIG. 7A). A small piece of Kapton Tape 186 was used to secure the copper tape strip 184 at the contact point to avoid any contact resistance as shown in FIG. 8B.

The resistance of the sensor was 1.2Ω before the contact, and after making contacts with the copper tape, the resistance remained the same. This shows that a zero-contact resistance can be achieved by simply using the conductive adhesive of the copper tape, eliminating further need for soldering and epoxy. Similarly, the paper humidity sensor 188 was also converted into modular form as shown in FIG. 8C.

Then, a sticker was prepared, which would serve as both a carrier platform to host the modular sensors, as well as the base substrate onto which interconnects are formed, enabling conformal connections between the modular paper sensors and the central electronic interface 160. To prepare the base structure 181 onto which sensors would be attached (here onwards called a “sticker”), a Polyimide sheet (DuPont™ Kapton® 200HN Polyimide Film, 50 Micron Thickness) was selected (other materials may be used) and a pattern of interconnects 183 was prepared, using conductive copper tape as per the dimensions of the previously made modular paper sensors, as shown in FIG. 8D. Then, a Z-axis conductive tape (3M™ Z-Axis Conductive Tape 9703) 185 was placed on the area where the paper sensors needed to be attached. This Z-axis conductive tape conducts current anisotropically when two conductors are adhered firmly on the top and bottom sides of it. A zoom-in of the area shows that the conductive tape has embedded gold granules all over, allowing the anisotropic conduction of electric current. This is an advantageous property for the modular approach, deemed to be necessary to achieve the desirable zero Ohm contact resistance between the copper tape of the paper sensors and the copper tape on the polyimide sticker. The paper sensors 182 and 188 are then attached to the substrate 181, as shown in FIG. 8E, so that the conductive strips 184 of the sensors are mechanically and electrically connected to interconnects 183.

After assembly of the sensors 182 and 188 onto the substrate 181 in a completely modular fashion, the environment sensor 180 was obtained (see FIG. 8F) as a customized and flexible decal to monitor humidity and temperature inside the pill container. The environment sensor 180 is attached to the central electronic interface 160, as already shown in FIG. 7B. This process is fairly simple and requires minimal technical expertise by circumventing conventional techniques of soldering and etching used in Printed Circuit Boards, or microfabrication processes of metal deposition on Polyimide sheets used in a cleanroom. This means that the control system 120 discussed herein may be made by a person interested in electronics, without the need of a factory manufacturing process, which is expensive. In other words, the process discussed herein can be performed at home, if so desire by an user. Those skilled in the art would also understand that a company that intends to manufacture this control system can manufacture these sensors in a dedicated factory, using robots and all the available techniques known in the art.

An overview of how each sensor discussed above interfaces with the microprocessor 162 is now discussed with reference to FIG. 9. The diagram in FIG. 9 illustrates various components of the control system 120. The control system 120 includes the microprocessor 162 (also called a PSoC), the pill counter mechanism 130, the electronic interface 160, the strain sensor 142, the tamper sensor 150, and the environment sensor 180. FIG. 9 shows that microprocessor 162 is programmed to have a module that acts as a first current source 902, another module that acts as a second current source 904, a third module that acts as an operational amplifier 906, a fourth module that acts as an analog voltage sensing 908, a fifth module that acts as a pill counter logic 910, a sixth module that acts as a humidity sensing logic 912, and a seventh module that acts as a BLE module 916. A memory 914 is also present in the microprocessor 162.

The first current source 902 is connected to the strain sensor 142 and generates a current. The operational amplifier 906 measures the current and also a current that flows through a resistor R1 connected to ground. These readings are combined and provided to the analog voltage sensing module 908, which calculates whether a strain is applied to the strain sensor. The calculated value is provided to the memory 914 for storage and also to the BLE module 914, if the microprocessor decides to send out an alert.

The second current source 904 is connected to the temperature sensor 182 and sends a current through the sensor. A value of the measured current is sent to the analog voltage sensing for evaluating a voltage across the sensor. The value is then mapped to a corresponding temperature, which is stored in the memory 914.

The humidity sensing module 912 senses a voltage across the capacitive sensor 188 and calculates a humidity associated with the sensed voltage. Note that the sensed voltage depends on the dielectric of the capacitive sensor, which is influenced by the humidity around the sensor.

FIG. 9 also shows the pill counter module 910, which exchanges data with the pill counter mechanism 130 (i.e., the voltages shown in FIGS. 4A to 4D). Based on these voltages, the pill counter module 910 determines whether a pill is leaving or entering the container. This data may also be stored in the memory 914.

BLE module 916 is instructed by the microprocessor 162 when to send out data and what kind of data to send. The data is fetched from the memory 914. The data may be sent to any BLE-enabled device 920, for example, a smartphone of the patient. Those skilled in the art would understand that other configurations may be used.

To facilitate rapid transition of lab based innovation to technology and thus to empower everyone (any age group with any educational background and economic status), the approach discussed above was to formulate a complete application which can be easily integrated into current healthcare systems, and be able to make an impact on people's lives at a smaller price.

A central element of any control system for monitoring the dispense of pills is an optimal sensor interface with low-power consumption, data transmission capability, and a small footprint. In order to save power and keep a small footprint, the electronic interface of the above embodiments was assembled using only one IC, which was only possible because of the state of the art breakthrough in CMOS industry to produce Programmable System on Chip (PSoC). A Cypress® PSoC 214009 which has a 48 MHz microcontroller and a 256 kB Flash memory in a footprint of just 10×10 mm has been used. However, other microcontrollers may be used. All the sensors including paper humidity sensor, paper temperature sensor, and the voltage output from pill counter, were directly connected to the microcontroller without the use of any additional sensing interface. Wireless data transmission has a significant powerhead. Wired transmission consumes less power but limits the functionality of portable devices. With the introduction of latest BLE wireless transmission protocol, the devices are less power hungry when using a wireless transmission method. The Cypress® PSoC 214009 also possesses this BLE capability with PCB trace antenna on-board. The BLE module may be used for a 2-way communication, which includes sending a Bluetooth notification when it is time to take the pill, and then count the number of pills extracted. An application on the smartphone can serve various other purposes, like display side effects of the pills being taken, recommended food intake with the pill, expiry date, as well as temperature and humidity levels inside the container.

Another advantage of the embodiments discussed above is the flexibility of the control system. Because a flexible substrate (Polyimide) and flexible sensors (Paper and rubber) are used, and they are integrated together using anisotropic conductive tapes, it is possible to design the whole system in any desired shape.

The microcontroller shown in FIG. 9 advantageously uses less energy. Conventionally, to find changes of a resistance, a Wheatstone bridge is used, which employs 4 resistors connected to the battery separately, apart from the rest of the electronic interface. This arrangement consumes a lot of power, and thus, a bigger sized battery is needed for long-term operation. The current analog to digital converter (IDAC) component of the microcontroller 162 was programmed to act as a current source 902 and 904 in FIG. 9. This current source periodically feeds a fixed amount of current directly into the temperature sensor or strain sensor, and then the subsequent voltage is read by the Analog to Digital Converter (ADC) in the same microcontroller. Due to the ability to control the amount of current and feed current only when data are logging, the embodiment of FIG. 9 reduced the power requirement significantly. For the temperature sensor to be used in a Wheatstone bridge and powered by a 3.7V battery, the continuous current draw was about 470 mA. However, the embodiment of FIG. 9 fed only 300 μA of current into the temperature sensor and only when needed, i.e., once a second, which equates to energy savings of about 99.99%.

Temperature measuring resistive sensors have a small resistance corresponding to only small voltage changes, which consequently results in reduced sensitivity. Thus, an Amplifier IC module 906 may be used to enhance their sensitivity, which draws a power of around 15-40 mA, in addition to the power consumed by the Wheatstone bridge and the electronic interface. The microcontroller 162 increased the sensitivity to 4.74 mV/° C. when the internal Operational Amplifier 906 was used.

To monitor the humidity inside the prescription bottle, a paper based humidity sensor is used. This sensor, as reported by Nassar et al., changes its capacitance in response to changes in humidity. In order to monitor changes in capacitance, generally, a Capacitance to Digital converter (CDC) IC is needed for the microcontroller to be able to sense the values of capacitance. This consumes extra power and space. However, the microcontroller 162 was programmed to use the inbuilt CapSense® feature to measure the capacitance without the need for any external components. Capacitance changes detected by the microcontroller are mapped into humidity changes. A threshold can be set, past which the system generates an alert that the humidity levels have gone past the limit. The responsivity of the sensor with the electronic interface came out with 640 ms rise time (T_{R}) and 540 fall time (T_{F}), which is fast enough to report sudden humidity changes inside the container.

Experiments have also shown that when the hand is brought close to the electronic interface with the tamper sensor attached, the capacitance decreases slightly due to proximity effects. For this test, the hand was hovering 2 cm above the tamper sensor. The moment the hand touches the surface of the sensor, the system detects the pressure, and the capacitance increases significantly due to a decrease of the parallel plate spacing because of the applied pressure.

The strain sensor attached to the walls of the bottle is a resistive sensor changing its resistance when subjected to strain. This concept was used to measure any forced entry attempt on the bottle. This strain sensor shares the same interface as the paper heat sensor. A small amount of current is injected into the resistive sensor and the system measures the voltage accordingly. The voltage output from the strain sensor is sensed by the electronic interface and experiments have indicated the presence of snippets on the graph showing the change in the resistance value when the bottle is pressed and released. Similar results are obtained when the cap is removed, as the pull on the strain sensor changes the resistance of the sensor. A threshold voltage level can be set, above which an alert can be generated by using either Bluetooth, logging into the memory, lighting up an LED or sending a SMS using a GSM module.

Using low-cost paper sensors and a flexible decal electronic interface, it is possible to use a modular integration strategy to present a complete solution to the serious problem of drug overdose. The assembled system produced reliable and consistent results. The integration process involves assembly of low-cost materials without the need of complex fabrication processes. The devices can be easily installed and removed from a customizable 3D printed cap on a prescription container. The system has the capabilities of counting pills going in and out of the sensor, detecting forced entry attempt on the container, detecting electronic interface tampering attempts, monitoring temperature and humidity inside the container, and sending alerts using Bluetooth and buzzer/LED.

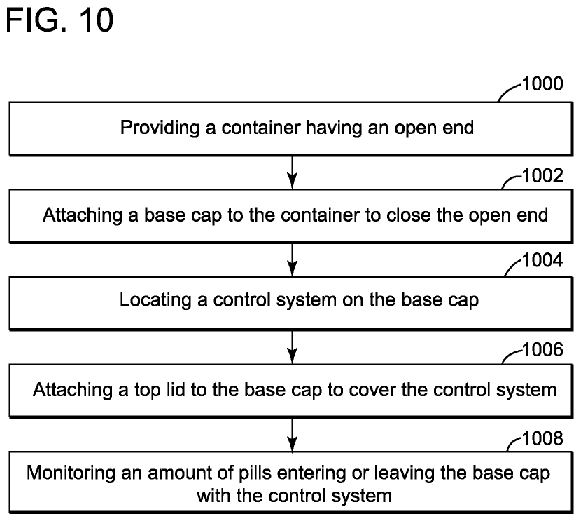

A method for dispensing a drug with one of the systems discussed above is now discussed with regard to FIG. 10. The method includes a step 1000 of providing a container having an open end, a step 1002 of attaching a base cap to the container to close the open end, a step 1004 of locating a control system on the base cap, a step 1006 of attaching a top lid to the base cap to cover the control system, and a step 1008 of monitoring with the control system an amount of pills entering or leaving the base cap.

The disclosed exemplary embodiments provide methods and systems for monitoring a product entering or leaving a container. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

Safd J. M. Nassar, M. M. Hussain, IEEE Transactions on Electron Devices 2017, 64, 5.
J. M. Nassar, K. Mishra, K. Lau, A. A. Aguirre-Pablo, M. M. Hussain, Advanced Materials Technologies 2017, 2, 4
J. M. Nassar, M. D. Cordero, A. T. Kutbee, M. A. Karimi, G. A. T. Sevilla, A. M. Hussain, A. Shamim, M. M. Hussain, Advanced Materials Technologies 2016, 1, 1.
J. P. Rojas, G. A. Torres Sevilla, M. T. Ghoneim, S. B. Inayat, S. M. Ahmed, A. M. Hussain, M. M. Hussain, ACS Nano 2014, 8, 2
G. A. Torres Sevilla, M. T. Ghoneim, H. Fahad, J. P. Rojas, A. M. Hussain, M. M. Hussain, ACS Nano 2014, 8, 10.

1. A control system for monitoring drug dispensation from a container the system comprising:
a base cap configured to be attached to the container;
an electronic interface attached to the base cap;
a microprocessor attached to the electronic interface;
a pill counter mechanism attached to the base cap and configured to count pills that are passing the base cap; and
a top lid that attaches to the base cap and fully encloses the electronic interface, the microprocessor and the pill counter mechanism.

2. The system of claim 1, wherein the pill counter mechanism includes a laser emitting diode and a photodiode for detecting the passing of the pills.

3. The system of claim 1, further comprising:
a tamper sensor located over the microprocessor and connected to the microprocessor,
wherein the microprocessor determines from readings of the tamper sensor whether access to the microprocessor has been tried.

4. The system of claim 1, further comprising:
a temperature sensor connected to the electronic interface; and
a humidity sensor connected to the electronic interface,
wherein the microprocessor determines a temperature and humidity inside the container from readings of the temperature sensor and the humidity sensor.

5. The system of claim 4, wherein the temperature sensor and the humidity sensor are located outside the base cap and the top lid.

6. The system of claim 5, wherein the base cap has a slot, and wherein a substrate to which the temperature sensor and the humidity sensor are attached, extends from inside the container, through the slot, to a chamber defined by the base cap and the top lid.

7. The system of claim 6, wherein the substrate, the temperature sensor and the humidity sensor are flexible.

8. The system of claim 4, wherein the temperature sensor and the humidity sensor are paper based sensors.

9. The system of claim 1, wherein the base cap has a slot and a slide located around the slot to guide the pills in and out of the container.

10. The system of claim 9, wherein the pill counter mechanism is attached to corresponding grooves formed in the slide.

11. The system of claim 1, further comprising:
a strain sensor attached to a wall of the container and electrically connected to the electronic interface.

12. The system of claim 1, further comprising:
a battery attached to a slide formed in the base cap.

13. The system of claim 1, wherein the base cap has a slide and the top lid has a slot that communicates with the slide to allow the drug to get in and out of the container.

14. A drug dispensing system comprising:
a container having an open end;
a base cap configured to be attached to the container and close the open end;
a control system located on the base cap; and
a top lid attached to the base cap and covering the control system,
wherein the control system is configured to monitor a pill entering or leaving the container.

15. The system of claim 14, wherein the control system comprises:
an electronic interface attached to the base cap;
a microprocessor attached to the electronic interface; and
a pill counter mechanism attached to the base cap and configured to count the pill entering or leaving the base cap.

16. The system of claim 14, further comprising:
a tamper sensor located over the microprocessor and connected to the microprocessor,
wherein the microprocessor determines from readings of the tamper sensor whether access to the microprocessor has been tried.

17. The system of claim 14, further comprising:
a temperature sensor connected to the electronic interface; and
a humidity sensor connected to the electronic interface,
wherein the microprocessor determines a temperature and humidity inside the container from readings of the temperature sensor and the humidity sensor.

18. The system of claim 14, wherein the base cap has a slot and a slide located around the slot to guide the pill in and out of the container and a pill counter mechanism is attached to corresponding grooves formed in the slide.

19. The system of claim 14, further comprising:
a strain sensor attached to a wall of the container and electrically connected to the electronic interface.

20. A method for dispensing a drug, the method comprising:
providing a container having an open end;
attaching a base cap to the container to close the open end;
locating a control system on the base cap;
attaching a top lid to the base cap to cover the control system; and
monitoring a pill entering or leaving the base cap with the control system.
